(19) United States
(12) Patent Application Publication
(10) Pub. No.: US 2008/0146946 A1
(43) Pub. Date: Jun. 19, 2008

(54) BLOOD PRESSURE SENSOR APPARATUS

(76) Inventors: Valentino Montegrande, Coto De Caza, CA (US); Kevin S. Montegrande, San Francisco, CA (US)

Correspondence Address: LAW OFFICES OF ERIC KARICH, 2807 ST. MARK DR., MANSFIELD, TX, 76063, US

(21) Appl. No.: 11/959,170

(22) Filed: Dec. 18, 2007

Related U.S. Application Data
(63) Continuation-in-part of application No. 11/374,575, filed on Mar. 13, 2006, now abandoned.
Application No. 11/374,575 is a continuation-in-part of application No. 10/812,588, filed on Mar. 29, 2004, now abandoned.
(60) Provisional application No. 60/458,660, filed on Mar. 28, 2003.

(51) Int. Cl. A61B 5/0215

(52) U.S. Cl. 600/486

(57) ABSTRACT

A method for measuring blood pressure utilizes an implantable sensor for measuring blood pressure. The implantable sensor includes a probe having a neck portion extending outwardly from the main body to a conical locking flange; a terminus of the conical locking flange forming an aperture that is covered with a flexible membrane that defines an internal chamber that is filled with a biocompatible fluid; and a capacitor electronically connected to the implant inductor and operatively positioned adjacent the internal chamber. The implantable sensor is positioned adjacent a blood vessel such that the probe extends through a blood vessel wall such that the conical locking flange lockingly engages the blood vessel wall.

### CROSS-REFERENCE TO RELATED APPLICATIONS

This application for a utility patent is a continuation-in-part of a previously filed utility patent, now abandoned, having U.S. Utility application Ser. No. 11/374,575, filed Mar. 13, 2006. That application was a continuation-in-part of a previously filed utility patent, also abandoned, having U.S. Utility application Ser. No. 10/812,588, filed Mar. 29, 2004. This application further claims the benefit of U.S. Provisional Application No. 60/458,660, filed Mar. 28, 2003.

### BACKGROUND OF THE INVENTION

1. Field of the Invention

This invention relates generally to a blood pressure sensor apparatus and methods, and more particularly to a method for sensing a blood pressure using an implantable sensor that extends through a wall of a blood vessel and functions to regularly report the blood pressure of the patient.

2. Description of Related Art

The monitoring of blood pressure by caregivers has become a well-characterized biomonitoring tool. Hypertension, hypotension, shock and circadian rhythm are some examples of conditions monitored via blood pressure. In most cases, the usage of a sphygmomanometer and a pressure cuff suffice. But in cases where long-term, mobile, non-tethered, and/or physician-free patient monitoring is required, a more elaborate and implantable system may be needed.

The foremost requirement for implantation is the size of the device. The implant should not impart any physiological disturbance nor should it present any substantial inconvenience. Furthermore, the device may only protrude into a blood vessel a very small amount, because the introduction of a significant disturbance into a blood vessel can cause health problems.

Supplying power to the device and rate of power consumption are also important factors because battery size and replacement are critical limiting factors to the miniaturization and operation of the device. Finally, a means of transmitting the signal is an integral part of the implant as well as a technique to encapsulate the entire device for the bilateral protection of the physiology and the implant.

Prior art devices are taught by Bullara, U.S. Pat. No. 4,127,110, which teaches an implantable pressure transducer for the wireless measurement of the pressure of various bodily fluids, particularly in the brain. The Bullara sensor includes an elongated cannula that may be inserted into a body cavity for the measurement of fluid pressures. While the Bullara device is generally similar in structure, it is not adapted to be lockingly mounted to a blood vessel. Allen, U.S. Pat. No. 7,147,604, teaches a sensor that is adapted to be mounted to tissue using anchors (shown in FIGS. 6 and 7); however, the anchors used in this sensor do not themselves transmit pressure from the tissue in which they are anchored, then merely penetrate and lock with the tissue.

While the prior art teaches many elements of the present invention, the prior art does not teach a probe having a neck portion extending outwardly from the main body to a conical locking flange, the conical locking flange having a diameter that is larger than the neck portion and being shaped to penetrate through and then lockingly engage the blood vessel wall; and a terminus of the conical locking flange forming an aperture that is covered with a flexible membrane that defines an internal chamber, the internal chamber being filled with a biocompatible fluid for measuring the pressure of the fluid within the blood vessel.

### SUMMARY OF THE INVENTION

The present invention teaches certain benefits in construction and use which give rise to the objectives described below.

The present invention provides a method for measuring blood pressure. The method comprising the steps of providing an implantable sensor, and surgically implanting the implantable sensor for measuring blood pressure in a blood vessel through a blood vessel wall. The implantable sensor comprising: a main body having an implant inductor; a probe having a neck portion extending outwardly from the main body to a conical locking flange, the conical locking flange having a diameter that is larger than the neck portion and being shaped to penetrate through and then lockingly engage the blood vessel wall; a terminus of the conical locking flange forming an aperture that is covered with a flexible membrane that defines an internal chamber, the internal chamber being filled with a biocompatible fluid; and a capacitor electronically connected to the implant inductor and operatively positioned adjacent the internal chamber for measuring pressure within the blood vessel by measuring the pressure of the biocompatible fluid. The implantable sensor is then positioned adjacent the blood vessel such that probe extends through the blood vessel wall and into the blood vessel such that the conical locking flange lockingly engages the blood vessel wall. A primary objective of the present invention is to provide a method for continually measuring blood pressure of a patient, the method having advantages not taught by the prior art.

Another objective is to provide an implantable sensor that can readily be positioned outside of a conduit such as a blood vessel without undue trauma to the patient, yet accurately measure the pressure within the blood vessel.

Another objective is to provide an implantable sensor that includes a probe that can be positioned through the blood vessel so that blood flow within the blood vessel is not significantly impeded or disrupted.

Another objective is to provide an implantable sensor that includes a probe having a neck portion extending outwardly from the main body to a conical locking flange, the conical locking flange having a diameter that is larger than the neck portion and being shaped to penetrate through and then lockingly engage the blood vessel wall; and a terminus of the conical locking flange forming an aperture that is covered with a flexible membrane that defines an internal chamber, the internal chamber being filled with a biocompatible fluid for measuring the pressure of the fluid within the blood vessel.

A further objective is to provide an implantable sensor that can be installed in a single procedure and then take continuous blood pressure measurements without further surgical procedures being required.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is a perspective view of one embodiment of a blood pressure sensor apparatus;

FIG. 2 is a sectional view thereof taken along line 2-2 in FIG. 1;

FIG. 3 is a block diagram thereof;

FIG. 4 is a bottom perspective view of an implantable sensor;

FIG. 5 is a side elevational view thereof, a portion of the implantable sensor being shown broken away to illustrate first and second electrodes;

FIG. 6 is a side elevational view thereof, a portion of the implantable sensor being shown broken away to illustrate a flexible membrane and a capacitive membrane in deflected positions;

FIG. 7 is a top perspective view of the implantable sensor illustrating a plurality of bores in a top surface of the implantable sensor;

FIG. 8 is a perspective view of the blood pressure sensor apparatus transmitting data to a personal transmitter/receiver that is operatively attached to a computer; and

FIG. 9 is a perspective view of the blood pressure sensor apparatus transmitting data through a cellular transmitter/receiver to a data center.

### DETAILED DESCRIPTION OF THE INVENTION

The above-described drawing figures illustrate the invention, a blood pressure sensor apparatus 10 and method for periodically measuring the blood pressure of a patient.

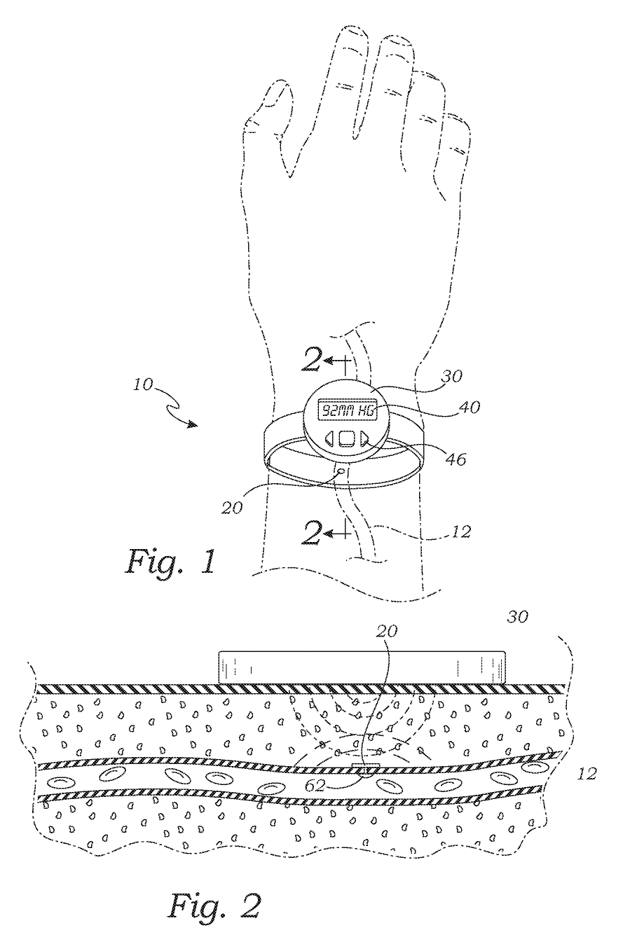

As shown in FIGS. 1-2, the blood pressure sensor apparatus 10 includes an implantable sensor 20 and an external reader 30. The implantable sensor 20 is adapted to be implanted in the patient for sensing the blood pressure. The external reader 30 is adapted to be positioned adjacent the implantable sensor 20, outside the body of the patient, and inductively coupled to the implantable sensor 20 to periodically read the blood pressure of the patient.

In the preferred embodiment, the external reader 30 is a wristwatch that can be conveniently worn by the user around his or her wrist. However, in alternative embodiments, the external reader 30 could be shaped to be worn around any portion of the body that is suitable for the implantable sensor 20. While it is currently preferred that the external reader 30 be adapted to be worn for significant periods of time, the external reader 30 could also be a hand-held scanner that is not worn, but is periodically positioned adjacent the patient to take blood pressure readings.

While we discuss the use of the blood pressure sensor apparatus 10 to measure the blood pressure of a patient, typically a human, the blood pressure sensor apparatus 10 can be used to measure the blood pressure in any animals, or indeed any closed system that includes a fluid flow whose pressure may be measured. Such alternative applications of the present apparatus should be considered within the scope of protection of the present patent.

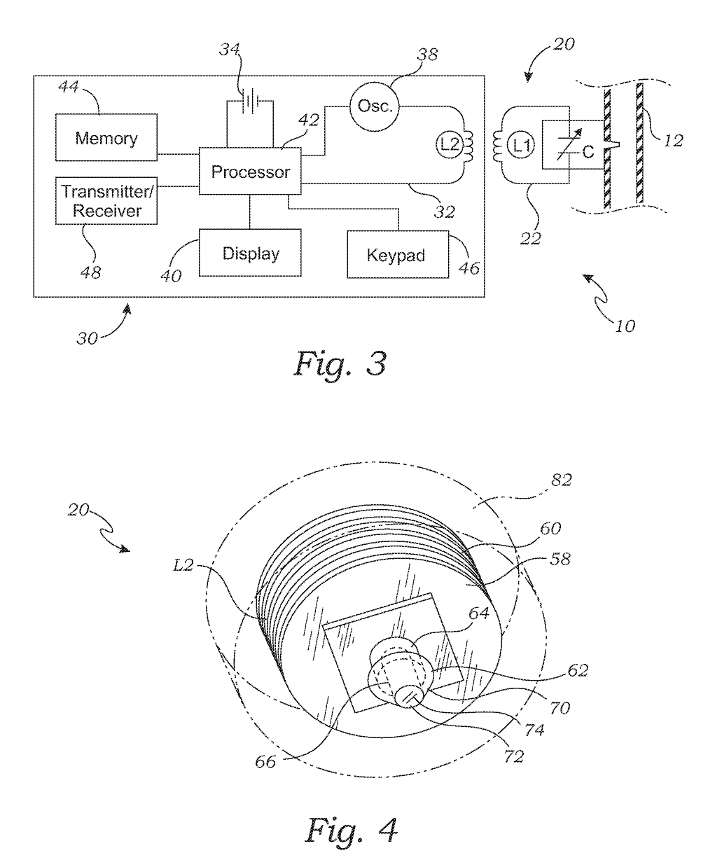

As shown in FIG. 3, the implantable sensor 20 includes an implant circuit 22 that includes a capacitor C electronically connected to an implant inductor LI. The external reader 30 includes an external circuit 32 that includes a power supply 34 electronically coupled to an external inductor L2 and to an oscilloscope 38. The oscilloscope 38 is adapted to perform a “grid-dip” sweep wherein the external reader 30 sweeps through a range of frequencies until it reaches a point that resonates with the implant circuit 22 and the oscilloscope measures a “dip.”. Since the frequency of resonance will vary depending upon the capacitance of the capacitor C, and thus the patient's blood pressure, it is possible to measure the blood pressure of the patient from the external reader 30 with reference to a simple calibration table.

The implant circuit 22 also includes a means for reporting the results of the “grid dip” sweep. In one embodiment, as shown in FIGS. 1 and 3, the external reader 30 includes a display 40, such as an LCD screen or similar feature, then enables the user to read the results of the measurements being taken. In this embodiment, the external circuit 32 includes a processor 42, a memory 44, and a keypad 46 for enabling the user to control the external reader 30. The inclusion of these additional elements enables the user to store multiple readings within the memory 44 for later review and/or download to a computer 52 using techniques well known in the art. Since the construction of such a circuit is well known to one skilled in the art, given the teachings of this invention, the specific construction of the external reader 30 is not described in greater detail herein.

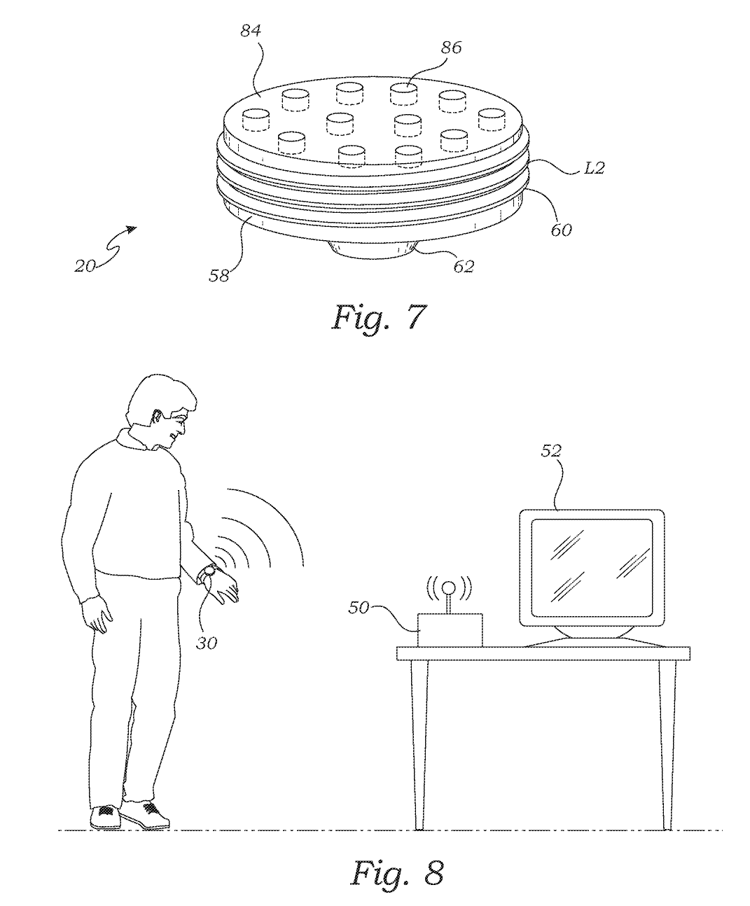

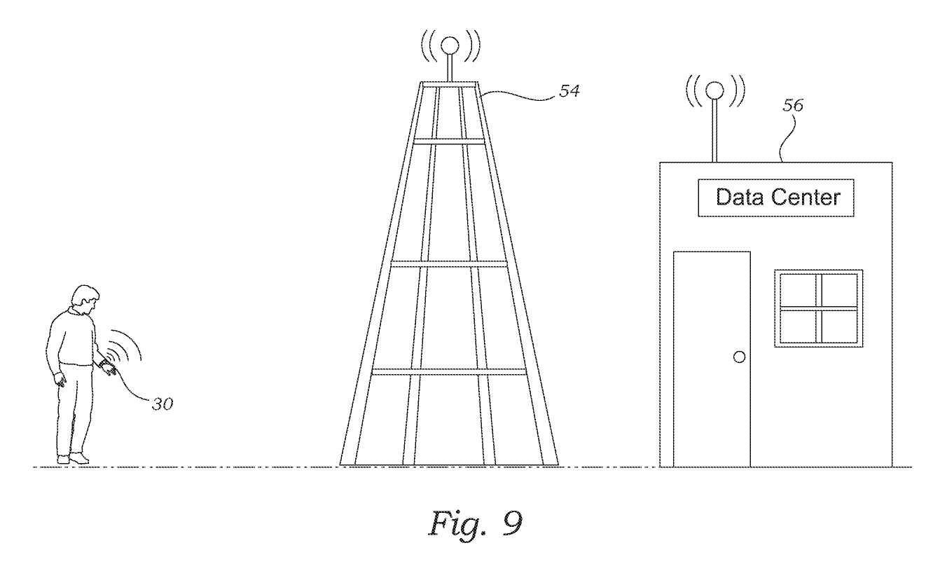

As shown in FIG. 3, the external reader 30 can also include a transmitter/receiver 48 for transmitting the measurements taken by the external reader 30. In one embodiment, shown in FIG. 7, the transmitter/receiver 48 transmits data to a personal transmitter/receiver 50 that is electronically connected to a computer 52. Upon a query from the computer 52, which could be located in a patient's home or in a doctor's office, the transmitter/receiver 48 of the external reader 30 could transmit the readings that were taken previously and stored in the memory 44.

In another embodiment, shown in FIG. 8, the transmitter/receiver 48 could transmit the data using cellular technology through a cellular transmitter/receiver 54 to a data center 56 for collection, analysis, and reporting. Obviously, many equivalent communications systems could be used, including satellite or IR transmissions, communications through a global computer network such as the Internet®, or a local area network. Any of these or similar reporting systems should be considered within the scope of the present invention.

Of course, communications between the external reader 30 and the computer 52 or the data center 56 would be two-way, thereby enabling many options in taking, reporting, and responding to blood pressure measurements. For example, if a patient's blood pressure were to get so high or so low as to threaten the health of the patient, and immediate warning could be sent to the patient, as well as the patient's doctor and/or a local ambulance dispatcher. The blood pressure sensor apparatus 10 could also be integrated with other systems, such as a medication injection device (not shown), that would automatically administer treatment in response to high or low blood pressure.

As shown in FIGS. 4-6, the implantable sensor 20 preferably includes main body 58 and a probe 62 that extends outwardly from the main body 58. The main body 58 includes the implant inductor LI and any other electronics or other useful structural features. In one embodiment, the main body 58 is generally cylindrical and the conductive material that forms the implant inductor LI formed in a coil around a perimeter 60. Due to the minimum size requirements of the implanted inductor LI, the main body 58 is adapted to remain outside the blood vessel 12 of the patient, thereby minimizing the potentially harmful impact of the implantable sensor 20 on the blood flow of the patient.

As shown in FIG. 6, the probe 62 is adapted to extend into the blood vessel 12 for the purpose of measuring the pressure in the blood vessel 12. The probe 62 must be small enough to prevent thrombosis or other health complications in the patient.

As shown in FIGS. 4-6, in the preferred embodiment, the probe 62 includes a neck portion 64 that extends outwardly to a conical locking flange 66. The neck portion 64 is preferably cylindrical and includes an internal saline chamber 68. The conical locking flange 66 is shaped to penetrate through and then lockingly engage the blood vessel 12. The conical locking flange 66 is preferably generally conical in shape, although this term is hereby defined to include equivalent shapes that have a diameter that is larger than the diameter of the neck. While one particular embodiment of the conical locking flange is disclosed, alternative structures may be devised by those skilled in the art that perform the same penetration/locking function, and the term conical locking flange is hereby defined to include these alternative structures that are equivalent thereto or that may be devised by those skilled in the art.

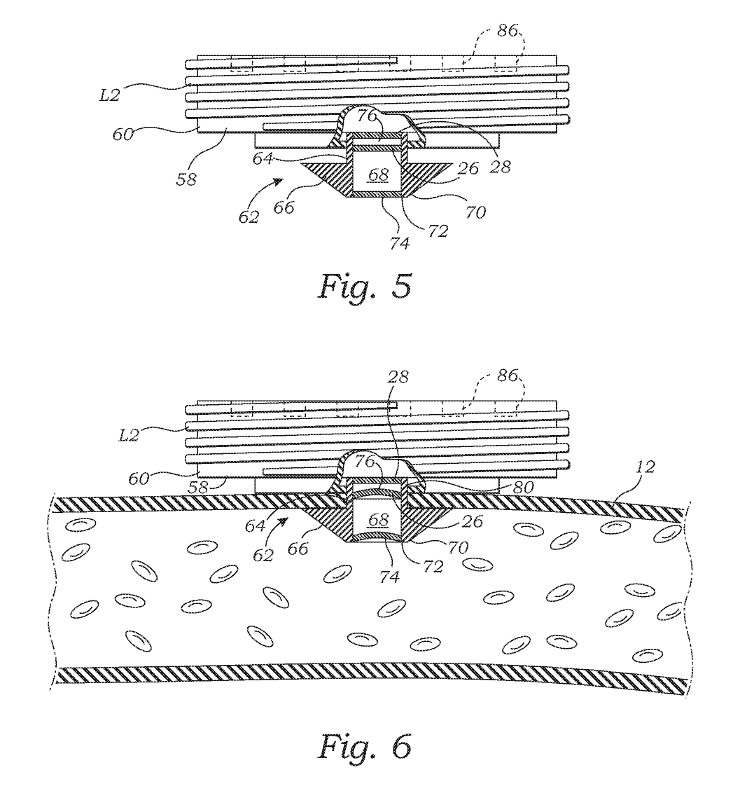

As shown in FIGS. 5-6, a terminus 70 of the conical locking flange 66 forms an aperture 72 that is covered with a flexible membrane 74. The internal saline chamber 68 is filled with saline or other biocompatible fluid or equivalent material that is contained within the internal saline chamber 68 by the flexible membrane 74.

In the embodiment of FIGS. 5-6, the capacitor may be formed of first and second electrodes 26 and 28. The first electrode 26 is preferably a flexible capacitive membrane that forms the 20 rear of the internal saline chamber 68 opposite the flexible membrane 74. The second electrode 28 is preferably a rigid capacitive membrane that is positioned a suitable distance from the first electrode 26, separated by a gap 76 that is suitable to form the capacitor C (of FIG. 3).

The first electrode 26 may be formed by a capacitive membrane formed of a highly doped silicon in conjunction with highly insulating support layers 80. The highly insulating support layers 80 are useful in limiting parasitic capacitance, which may otherwise interfere with accurate pressure measurement. Those skilled in the art can devise many alternative forms of the first electrode 26, and such alternative structures should be considered within the scope of the present invention.

In operation, pressure from the blood vessel 12 causes a deflection of the flexible membrane 74, which is transmitted through the saline (or other biocompatible fluid) in the internal saline chamber 68 to the flexible capacitive membrane 26, which in turn is deflected. As shown in FIG. 6, when the capacitive membrane 26 is deflected, this changes the size of the gap 76 between the capacitive membrane 26 and the rigid capacitive membrane 28 (which does not deflect), thereby altering the capacitance of the capacitor C. Changes in the capacitance cause a change in the frequency at which the external reader 30 measures a “dip” in the oscilloscope 38, as described above.

The conical locking flange 66, shown in FIGS. 4-5, is adapted to facilitate the penetration of the probe 62 through a vessel of the patient so that the flexible membrane 74 is positioned inside the blood vessel 12, as shown in FIG. 2. The neck portion 64 is adapted to extend through the blood vessel 12 so that the main body 58 is located outside the blood vessel 12, thereby minimizing any interference that the implantable sensor 20 may cause within the blood vessel 12. The flexible membrane 74 is disposed on an outside surface 78 of the implantable sensor 20 so that the flexible membrane 74 is exposed to the patient's blood once the implantable sensor 20 has been implanted in the patient.

The implantable sensor 20, and the capacitive membrane 26, are preferably constructed of silicon and formed using MEMS manufacturing techniques known in the art. By utilizing MEMS construction techniques, the implantable sensor 20 can be made extremely small, thereby minimizing the problems that can occur when a sensor is implanted in a patient's body. In one embodiment, as shown in FIG. 4, the implantable sensor 20 can be coated with a biocompatible coating 82, or housed within a suitably biocompatible structure, to prevent biocompatibility problems once the implantable sensor 20 has been implanted into the patient. The biocompatible coating 82 may also include embedded anti-coagulants (not shown) that are released throughout the intended lifetime of the sensing unit.

As shown in FIG. 6, an upper surface 84 of the implantable sensor 20 may include a plurality of bores 86 or “bosses.” The plurality of bores 86 function to increase the signal and improve the linear response. The plurality of bores 86 are preferably evenly spaced to increase their effectiveness.

Alternative Sensor Means

While the inductor/capacitor system that is described herein is currently the preferred sensor means, alternative sensor means (not illustrated herein) could also be utilized. For example, the sensor means could be provided by a piezoelectric sensor, a strain gauge, or another sensor known to those skilled in the art.

These alternative sensor means could be powered by the inductor system described above, be miniature batteries operably installed in the main body 58 of the implantable sensor 20, or by a resonant circuit that receives power from an external signal and then returns a return signal that reports a reading taken by the sensor means. Such alternatives should be considered within the scope of the present invention.

Method of Implantation and Use

The implantable sensor 20 is preferably to be implanted in the distal antebrachial region (forearm) adjacent the Ulnar or Radial arteries, since the thickness of integumentary tissues is relatively and consistently thin across this portion of the body. This site will also permit for easy placement of the external reader 30, in the embodiment of a wristwatch. Of course, those skilled in the art could devise alternative locations for the implantation and monitoring of the implantable sensor 20, and placement in an alternative location should be considered within the scope of the present invention.

The implantable sensor 20 preferably utilizes the passive system described above to eliminating any in-vivo power source requirement. The capacitive sensor system described above measures blood pressure by measuring the deflection of the capacitive membrane 26 that provides one electrode of a capacitive pair. The pressure sensor capacitance is part of an electrically resonant LC circuit load where L represents inductance and C represents capacitance. An alternating signal generated by the external reader 30 is transmitted at various frequencies to ‘sweep’ a response from the implant passive circuit. The transmitted input signal is coupled into the passive circuit at the LC resonant frequency, f, determined by:

$$f = \frac{1}{2 π} \frac{1}{\sqrt{LC}}$$

There is a non-ideal resistance, R, in the LC passive circuit that degrades the resonance response. Along with the membrane deflection with pressure, the quality factor, Q, is a measure of the device sensitivity and is given by:

$$Q = \frac{2 π fL}{R}$$

The objective is to design the implant circuit 22 with minimum resistance. Coil design, material selection, and interconnection to the pressure sensor are areas where minimal resistance is a critical design parameter.

If the capacitive membrane 26 is 1 mm×1 mm with a 1 um gap 76, the capacitance is approximately equal to 8.8 picofarads. A realizable mini-inductor can approach 1 microHenry. These values then estimate that the electronic detection circuit will operate in the vicinity of 50 mHz.

Sufficient pressure sensitivity and inductance can be housed in an implantable sensor 20 with dimensions roughly 5 mm in diameter and 0.3 mm in thickness. A small die size conflicts with larger membranes and inductor coils for greater sensitivity and lower “tank” frequency. (Inductance is inversely proportional to the square of the frequency.) The sensitivity of the sensor is governed by the flexibility of the capacitive membrane 26. A thin capacitive membrane 26 of large width provide the greatest sensitivity but can lead to nonlinearity problems. This effect is caused by the introduction of tensile stresses in the capacitive membrane 26 under load. Specialized “bossed” geometries, described above and in FIGS. 4-5, can be implemented for improved linear response.

Careful attention must be made to the electrical properties of the sensor structure. Since capacitance change is the measured property, the overall parasitic capacitances, Cp within the system must be kept at reasonable levels to obtain adequate sensitivity. For a capacitive signal-detecting circuit, the greatest sensitivity is achieved by maximizing the factor:

$$\frac{1}{{C}_{x} + {C}_{0} + 2 {C}_{p}} \frac{\partial ( {C}_{x} - {C}_{0} )}{\partial P}$$

where Cₓ is the capacitor C sensitive to the pressure, P. The reference capacitor C is designated by C₀. Capacitive membrane 26 materials such as highly doped silicon in conjunction with highly insulating support layers 80 can effectively limit the parasitic capacitance.

One of the key challenges is the accessibility of the blood to the pressure sensor. Due to the small size of the 3 mm diameter vessels, it is imperative that the implantable sensor 20 be as small as possible in order to facilitate insertion, minimize flow impedance and prevent thrombosis. Thus, the use of the probe 62 to extend into the blood vessel 12 while leaving the implantable sensor 20 outside the vessel solves many problems. This approach addresses issues concerning flow impedance, deployment, retrieval, and arterial embolism due to sensor detachment.

To avoid occlusion, the tip of the cannula can be capped off with a flexible membrane 74 so that pressure is translated across the membrane to a saline solution column on the opposite side. This design will communicate the pressure to the sensor external to the artery.

While the invention has been described with reference to at least one preferred embodiment, it is to be clearly understood by those skilled in the art that the invention is not limited thereto, but includes all similar, equivalent, or obvious alternatives that could be devised without undue experimentation by one of reasonable skill in the art.

What is claimed is:

1. A method for measuring blood pressure in a blood vessel through a blood vessel wall, the method comprising the steps of:
providing an implantable sensor comprising:
a probe having a neck portion extending outwardly from a main body to a conical locking flange, the conical locking flange having a diameter that is larger than the neck portion and being shaped to penetrate through and then lockingly engage the blood vessel wall;
a terminus of the conical locking flange forming an aperture that is covered with a flexible membrane that defines an internal chamber, the internal chamber being filled with a biocompatible fluid; and
a capacitor operatively positioned adjacent the internal chamber for measuring pressure of the biocompatible fluid;
positioning the implantable sensor adjacent the blood vessel;
penetrating the conical locking flange through the blood vessel wall such that probe extends through the blood vessel wall and into the blood vessel, and such that the conical locking flange lockingly engages the blood vessel wall; and
measuring the pressure within the blood vessel with the capacitor based upon the movements of the flexible membrane.

2. The method of claim 1, wherein the capacitor includes a flexible capacitive membrane in contact with the biocompatible fluid, and a rigid capacitive membrane spaced from the flexible capacitive membrane by a gap.

3. A method for measuring blood pressure in a blood vessel through a blood vessel wall, the method comprising the steps of:
providing an implantable sensor comprising:
a probe having a neck portion extending outwardly from the main body to a conical locking flange, the conical locking flange having a diameter that is larger than the neck portion and being shaped to penetrate through and then lockingly engage the blood vessel wall;
a terminus of the conical locking flange forming an aperture that is covered with a flexible membrane that defines an internal chamber, the internal chamber being filled with a biocompatible fluid; and
a capacitor electronically connected to the implant inductor and operatively positioned adjacent the internal chamber for measuring pressure within the blood vessel by measuring the pressure of the biocompatible fluid,
wherein the capacitor includes a flexible capacitive membrane in contact with the biocompatible fluid, and a rigid capacitive membrane spaced from the flexible capacitive membrane by a gap;
positioning the implantable sensor adjacent the blood vessel;
penetrating the conical locking flange through the blood vessel wall such that probe extends through the blood vessel wall and into the blood vessel, and such that the conical locking flange lockingly engages the blood vessel wall; and
measuring the pressure within the blood vessel based upon the movements of the flexible membrane and the induced movement of the flexible capacitive membrane in relation to the position of the rigid capacitive membrane.
